(19) United States
(12) Patent Application Publication
(10) Pub. No.: US 2014/0081362 A1
(43) Pub. Date: Mar. 20, 2014

(54) Implantable Medical Stimulator Lead With A Deployable Array Element And Method Of Use

(71) Applicant: Steve Wicklund, Scottsdale, AZ (US)

(72) Inventor: Steve Wicklund, Scottsdale, AZ (US)

(73) Assignee: Steve Wicklund

(21) Appl. No.: 13/621,057

(22) Filed: Sep. 15, 2012

(51) Int. Cl. A61N 1/05

(52) U.S. Cl. 607/116

(57) ABSTRACT

Stabilizing array, which includes a body elongating the distal tip of an implantable cylindrical stimulator lead, a storable, deployable and retroflexing array element for stabilizing distal tip of said lead, a keeper for storing array element during implantation, and a deploying lumen within the body which accepts a deploying stylet. The invention is a refinement to the prior art of a cylindrical stimulator lead. The array element functions to minimize migration of permanently placed cylindrical stimulator leads.

### REFERENCES CITED

U.S. PATENT DOCUMENTS

| | | | |
|---|---|---|---|
| 4,285,347 | August 1981 | Hess | 128/785 |
| 2005/0096718 A1 | May 2005 | Gerber et al. | 607/117 |
| 2006/0089692 A1 | April 2006 | Cross et al. | 607/116 |
| 7,099,718 B1 | August 2006 | Thacker et al. | 607/117 |
| 7,640,064 B2 | December 2009 | Swoyer | 607/115 |
| 2011/0022143 A1 | January 2011 | North | 607/117 |
| 7,891,085 B1 | February 2011 | Kuzma et al. | 29/825 |

### OTHER PUBLICATIONS

Reina M A. Electron microscopy and the expansion of regional anesthesia knowledge. Techniques in Regional Anesthesia and Pain Management, Vol 6, No. 4 (October), 2002: pp 165-171
Andrès J. Epidural space and regional anesthesia. European Journal of Pain Supplements, 3 (2009): pp 55-63
Anderson J M. Inflamatory response to implants. ASAIO 1988; 11:101-107.
Anderson J M. Biological responses to materials. Annual Reviews Material Research 2001; 31:81-110.
Attorney, Agent, or Firm—R. Joseph Trojan, Esq.
Attorney Docket: 12-09-6445

### TECHNICAL FIELD

The invention relates to implantable medical devices, more particularly, implantable medical leads.

### BACKGROUND

Spinal cord stimulation is used as an analgesic in patients with chronic and refractory pain syndromes and has had success in the treatment of neurogenic bladder syndrome.

Fundamentally a spinal cord stimulator consists of individually wired stimulator electrode contacts forming an electrode array which is incorporated into an implantable cylindrical or paddle lead. Stimulator electrode contacts are energized by a programmed stimulation sequence from a battery powered implantable pulse generator. To complete the circuit, the extra-epidural lead segment, which may require lead extensions, is tunneled under soft tissue where it plugs into the implantable pulse generator. Each medically implanted device is highly engineered for the integration of electronic components, coaxial porting and the use of durable biocompatible materials.

A cylindrical stimulator lead is introduced into an individual by means of a percutaneous technique whereas the paddle lead is introduced after performing a more invasive laminotomy procedure. Both lead types are implanted within the epidural space and the electrode array is positioned over a specific and targeted region of the spinal cord known as the dorsal column.

The positioning of the stimulator electrode array in a targeted location along the dorsal column is critical in determining the attenuation of chronic pain symptoms. Unfortunately lead migration, resulting in the loss of targeted dorsal column stimulation, is one of the common hardware related complications associated with spinal cord stimulator leads. This problem is associated more frequently with percutaneously placed cylindrical stimulator leads versus those of paddle designs. Patents are referenced for cylindrical stimulator leads that claim distal lead stability using glues, inflatable membranes, expanding wire loops, non-compliant loop-like elements and non-retroflexing tabs. Scar tissue formation into and round such elements may make retrograde removal of these leads difficult and potentially injurious to the contents of the epidural space. Furthermore, leads utilizing inflatable membranes to press against the epidural space have the potential for tissue ischemia and/or attenuated blood flow. Such leads may also limit the ability to place two stimulator leads side by side within the epidural space secondary to the space occupying volume of an expanded inflatable membrane.

As such, patients may benefit by having the electrodes placed by means of a percutaneous technique rather than having to undergo a more invasive laminotomy procedure, but the benefit only holds if the entire system is stable, safe and provides long term pain control.

### BRIEF SUMMARY OF THE INVENTION

In accordance with the invention, there is a need for a system of dorsal column stimulation using a percutaneously placed epidural cylindrical stimulator lead(s) that allows for long term fixed stability of said leads distally positioned stimulator electrode contacts. Described herein are methods for optimizing the stabilization of said leads stimulator electrode contacts with the use of a storable, deployable and retroflexing array element. Storage (folding) of the array element provides a means of percutaneous placement of said lead. Deployment of the array element, where it assumes its intrinsic shape, fixes the distal tip of said lead with a self-gripping mechanical interaction and inflammatory response which leads to scar formation and long term fixation.

Another purpose of the invention is provide a safe means of cylindrical stimulator lead removal utilizing a retrograde technique (prior art). A deployed array element has the ability to fold back on itself (retroflex) and assume a dimension no wider than any segment of the epidurally implanted cylindrical stimulator lead. Retroflexing of the array element upholds the practice of retrograde removal for the novel lead.

The invention relates to the refinement of a medically implantable cylindrical stimulator lead comprising: proximal and distal ends; individually wired stimulator electrode contacts on said leads distal end; wired contacts on said leads proximal end; and a coaxial lumen originating at said leads proximal tip, for receiving a guiding stylet.

The present invention relates, in one embodiment, to said cylindrical stimulator lead elongated distally by a stabilizing array comprising: a body continuous with the longitudinal axis said lead; a storable, deployable and retroflexing array element; a keeper (independent) of said stimulator electrode contacts for holding said array element in the stored (folded) position; a deploying lumen continuous with said coaxial lumen; contours on said body for accommodating said array element in stored and retroflexing positions; and radiopaque (x-ray) markers on said body and/or array element.

The present invention relates, in another embodiment, to said cylindrical stimulator lead elongated distally by a stabilizing array comprising: a body continuous with the longitudinal axis of said lead; a storable, deployable and retroflexing array element; a keeper (dependent) on an individual stimulator electrode contact for holding said array element in the stored position; a deploying lumen continuous with said coaxial lumen; contours on said body for accommodating said array element in stored and retroflexing positions; and radiopaque markers on said body and/or array element.

The present invention relates, in another embodiment to a stylet necessary for the deployment of the novel cylindrical stimulator leads array element comprising: a deploying stylet body; a control stop on said stylet body for contacting said leads proximal tip; and a deploying stylet integral with said stylet body, for insertion into said leads proximally originating coaxial lumen whereby advancement the deploying segment of said stylet, into said deploying lumen, induces the release of array element from the stored to deployed position.

The present invention relates, in yet another embodiment, to a deploying handpiece, utilized for retaining a proximal segment of said novel lead and deploying array element, generally comprising: a deploying stylet; a plunger integral with said deploying stylet; a cylinder which accommodates said plunger; a retention feature for securing a proximal segment of said lead; a seating surface to align said leads proximal tip; flexible tabs wherein the proximal end of said lead can be loaded and un-loaded from said retention feature; and a locking tab which prevents inadvertent deployment of said array element during percutaneous positioning of the novel cylindrical stimulator lead.

Henceforth, a cylindrical stimulator lead with the embodiment of said stabilizing array will be referred to as either the lead, novel lead, or cylindrical stimulator lead. Any further reference to the prior art of a cylindrical stimulator lead without said stabilizing array will be referred to as a non-stabilized lead or non-stabilized cylindrical stimulator lead.

### BRIEF DESCRIPTION OF THE DRAWINGS/FIGURES

The present invention is illustrated by way of example, and not by way of limitation, in the figures of the accompanying drawings and in which like references numerals refer to similar elements and in which:

FIG. 1 (prior art) illustrates the relevant components of an implanted medical stimulator;

FIG. 2 is the vertebral column illustrated in sagital, oblique and transverse partial dissections;

FIG. 2B, according to the present invention, illustrates the deployed array element of the cylindrical stimulator lead positioned within the epidural space;

FIG. 3A, according to the present invention, is a perspective view of a stabilizing array integral with a spinal cord stimulator lead;

FIG. 3B, according to the present invention, is a perspective view of a stabilizing array (variation) integral with a spinal cord stimulator lead;

FIG. 4, according to the present invention, are perspective views of independent and dependent keepers which may be used in the stabilizing array and said (variation) of FIG. 3;

FIG. 5 is an exploded perspective view of the stimulator lead and stabilizing array according to the present invention;

FIG. 6 is a partially assembled exploded perspective view of the stimulator lead and stabilizing array according to the present invention;

FIG. 7, according to the present invention, are perspective views of the stimulator lead and stabilizing array illustrating array element in deployed and stored positions;

FIG. 8A is a perspective axial cutaway view of the stimulator lead and stabilizing array according to the present invention;

FIG. 8B, according to the present invention, is a perspective axial cutaway view of the stimulator lead and stabilizing array illustrating a stored array element and a integrally formed keeper;

FIG. 9 is a perspective axial cutaway view of the stimulator lead and stabilizing array (variation) according to the present invention;

FIG. 10A, according to the present invention, is a perspective view of the stimulator lead and stabilizing array illustrating array element in the retroflexed position;

FIG. 10B, according to the present invention, is a perspective axial cutaway view of the stimulator lead and stabilizing array (variation) illustrating array element in the retroflexed position;

FIG. 11A, according to the present invention, is a perspective view of a stylet used in the deployment of array element of stabilizing array and said (variation);

FIGS. 11B and 14B (prior art) as pertaining to the present invention, shows a proximal segment of the stimulator lead;

FIG. 12, according to the present invention, are perspective axial cutaway views of the stimulator lead and stabilizing array illustrating the deployment of the array element;

FIG. 13 is a perspective view of the deploying handpiece according to the present invention; and

FIG. 14A is a top view of the deploying handpiece used in the deployment of array element of stabilizing array and said (variation).

### DETAILED DESCRIPTION OF THE INVENTION

The present invention will now be described in detail with reference to a few embodiments thereof as illustrated in the accompanying detailed description, examples, drawings and claims. Numerous specific details are set fourth in order to provide a thorough understanding of the present invention. However, before the present devices, systems, and/or methods are disclosed and described, it is to be understood that this invention is not limited to the specific devices, systems, materials, dimensions, and/or methods disclosed unless otherwise specified, and, as such, can, of course, vary. It will be apparent, however, to those skilled in the art, that the present invention may be practiced without some or all of these specific details. Furthermore, those who work in the art will recognize that many modifications and adaptations to the present invention are possible and can even be desirable in certain circumstances and are a part of the present invention. Thus, the following description is provided as illustrative of the principles of the present invention and not in limitation thereof. It is also to be understood that the terminology used herein is for the purpose of describing particular aspects only and is not intended to be limiting. In other instances, well known process steps have not been described in detail in order not to unnecessarily obscure the present invention. Given that the present invention is a refinement to the prior art of a non-stabilized cylindrical stimulator lead, a few references and figures pertaining to the prior art are made in this detailed description of the invention.

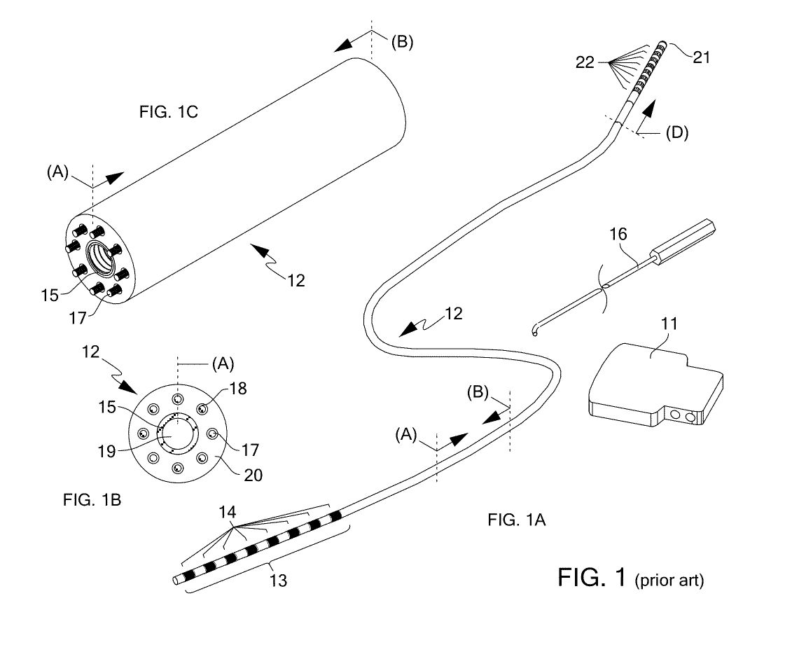

All of the details listed in FIG. 1 pertain to prior art and are included to aid in the description of the present invention. FIG. 1A is a perspective view indicating the relevant components of an implanted medical stimulator which comprise: a non-stabilized cylindrical stimulator lead 12; an implantable pulse generator 11; and a removable guiding stylet 16.

Wired contacts 22 at the proximal end of the non-stabilized lead 12 plug into implantable, battery powered, pulse generator 11 or lead extensions (not shown). Electrode array 13 is composed of independently wired stimulator electrode contacts 14 which are energized by output from implantable pulse generator 11.

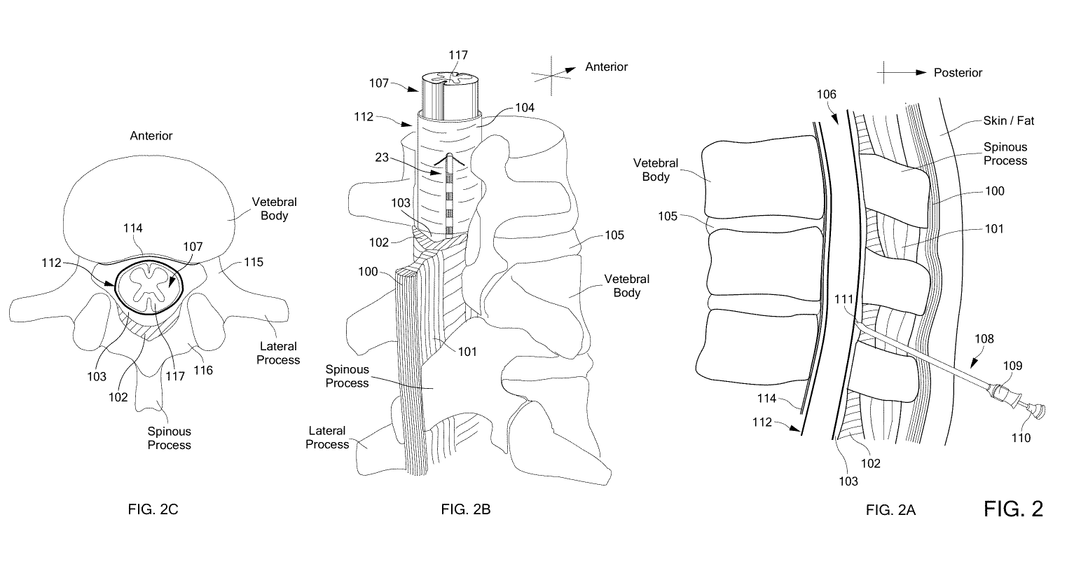

FIGS. 1B and 1C, cross-sectional and perspective views respectively, as taken from dissecting lines (A) and (B), show a characteristic depiction of the prior art as it pertains to insulating body 20 of non-stabilized lead 12. Eight wire conduit lumens 18 and electrical conductor wires 17, shown projecting from FIG. 1C, are depicted radially around coaxial lumen 19 which is encircled by stylet guide 15. Guiding stylet 16 is used to stiffen and direct non-stabilized lead 12, and novel lead 23, during percutaneous placement within the epidural space (103 of FIG. 2). With reference to FIG. 1, the illustrated components of non-stabilized lead 12, as taken from dissecting line (D) and those between dissection lines (A) and (B), remain unchanged for novel lead 23.

As illustrated throughout, and pertaining to prior art as referenced by Cross (US patent 2006/0089692 A1), stylet guide 15, of non-stabilized lead 12 and novel lead 23, is depicted as a coiled wire feature. In some instances stylet guide 15, as referenced by Cross and further by Kuzma (U.S. Pat. No. 7,891,085 B1), is a unique construct or may be omitted whereby insulating body 20 serves as stylet guide 15.

Within the human vertebral canal, as illustrated in FIG. 2, the epidural space 103 is both a true and potential space. The potential part becomes a true space when solutions or air are injected in it or, in this case, when cylindrical stimulator lead(s) (23 of FIG. 2B) is placed within it. The epidural space 103 is a cylindrical compartment surrounding the dural sac 112 of the spinal canal 106 and is further defined anteriorly by the posterior longitudinal ligament 114, and intervertebral discs 105, laterally by the vertebral pedicals 115 form the intervertebral foramina (not shown) while posteriorly it is delimited by the vertebral laminas 116 and the ligamentum flavum 102. The contents of epidural space 103 (not shown) includes nerve roots, connective tissue, variable amounts of fat and a venous plexuses. The external surface of the dural sac 112, composed of collagen and elastic fibers, is free and does not adhere to the vertebral canal.

The epidurally implanted distal segment of non-stabilized lead 12, which includes electrode array 13, has no inherent static or dynamic self-gripping elements, as such, non-stabilized lead 12 may move laterally and axially away from a targeted posterior midline position overriding the dorsal column (117 of FIGS. 2B and 2C) of the spinal cord 107. Such movement may be one factor resulting in attenuated or failure of therapeutic dorsal column 117 stimulation. Axial movement, as noted by Cross (US patent 2006/0089692 A1), occurs by tensile forces on non-stabilized lead 12 imposed by patient postural changes. Tensile forces may also cause lead failure, extra-epidural anchor damage (not shown), or tissue damage. Cross includes features in non-stabilized lead 12 constructs that increases the modulus of elasticity in an effort to lessen the impact of tensile forces. Of note, axial movement in the distal segment of non-stabilized lead 12 may be exaggerated by the accepted art of extra-epidural anchoring of said lead to soft tissue generally at the level of lead insertion into the epidural space 103.

The main purpose of the invention is to provide the distal segment of cylindrical stimulator lead 23 with fixed stability within the epidural space 103. By inference, fixed stability of the distal segment of lead 23 provides electrode array 13 stability over a targeted dorsal column 117 stimulation site which is significantly independent of patient postural changes.

Initial stability of the distal segment of lead 23 is achieved by a self-gripping array element 26 which, once deployed in a plane generally parallel to the posterior aspect of the epidural space 103, exerts resistance to axial and lateral movement by interacting with the connective tissues of the epidural space 103, including the collagen and elastic fibers of the dural sac 112. With reference to Reina and Andrès, scanning electron micrographs of the dural surface (dura) 104 show collagen and elastic fibers that are, to some extent, responsible for the initial securing the self-gripping feature of deployed array element 26.

Complementing the initial self-gripping interaction of deployed array element 26 is foreign body tissue inflammation and subsequent scar formation which encapsulates any permanent medical device. These processes have been assiduously characterized by Anderson et al. with respect to the general time course, cells involved, cell-cell interactions, and cell-biomaterial interactions. Within the epidural space 103, scar formation will chronically fixate the distal segment of lead 23 in axial and lateral planes, thereby reducing distal segment movement of lead 23 away from the targeted dorsal column 117 stimulation site.

The polymer selected for array element 26 will have the ability to return from a deformed state (temporary shape) to its intrinsic (permanent) shape. The permanent shape of array element 26 is the deployed configuration. The temporary (folded) shape is necessary for storage of the distal tips of array element 26 into keeper 27 and is mandatory for percutaneous placement of lead 23 within the epidural space 103. As illustrated and discussed below, a retroflexed position of array element 26 is also achievable if retrograde removal of lead 23 is necessary.

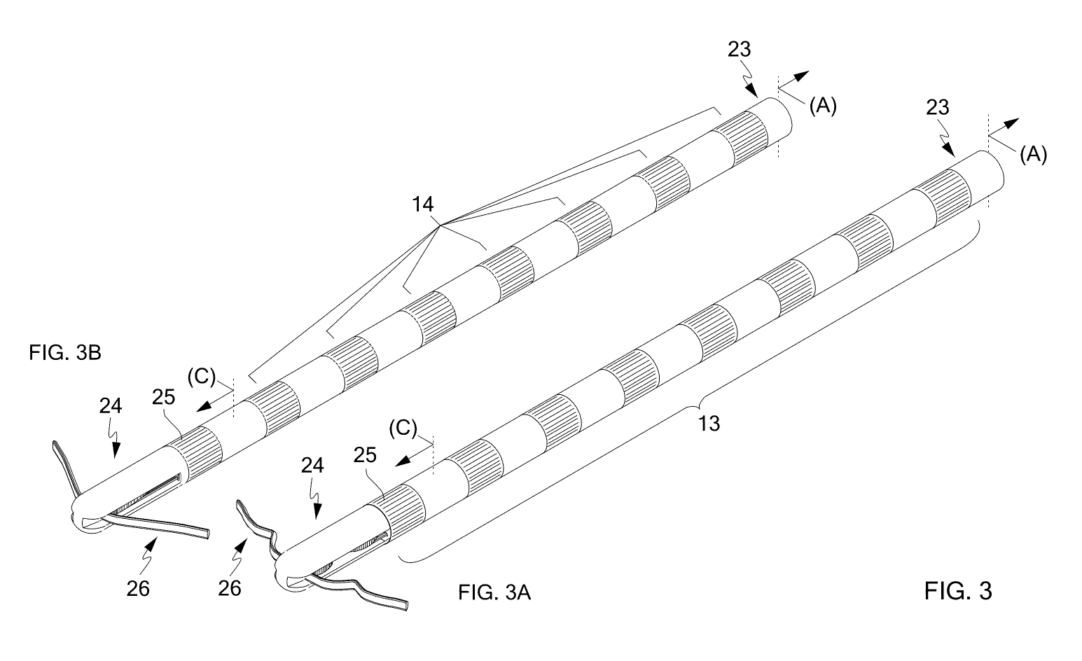

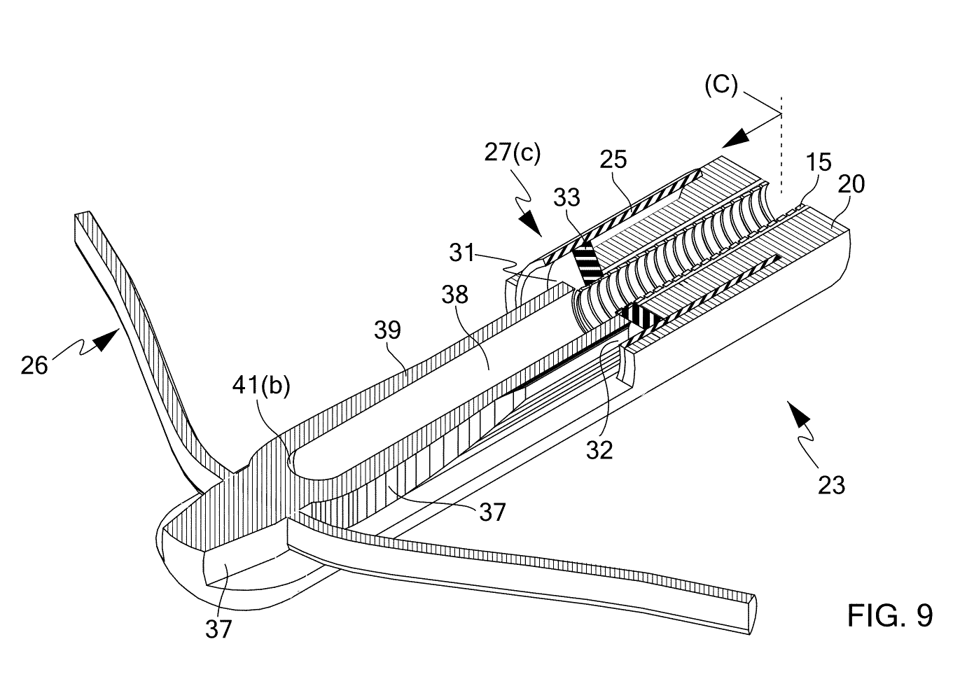

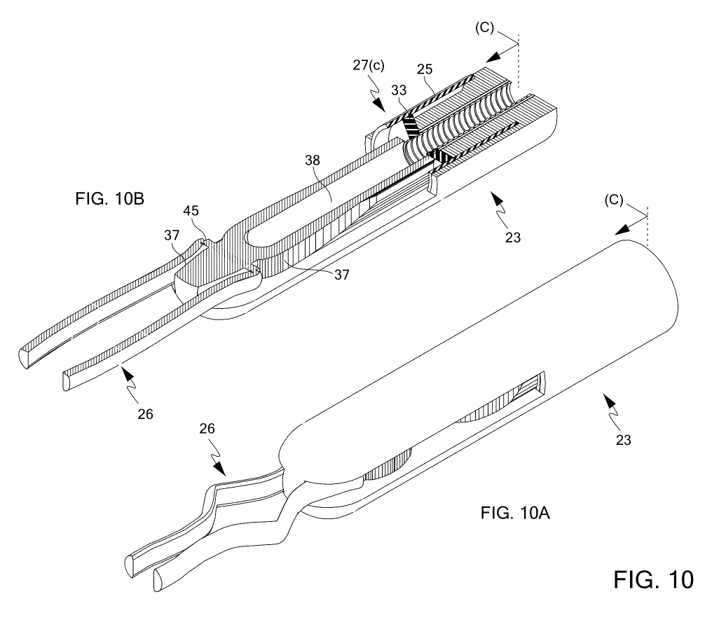

The deployed shape of array element 26 may be widely varied. It may, for example be somewhat rectilinear, curvilinear or a combination of the two. As an example, but not limitation, FIG. 3, shown in a perspective view, as taken from the distal segment of lead 23 across dissection line (A), depicts two embodiments of stabilizing array 24. In one embodiment, FIG. 3A, the deployed array element 26 is curvilinear, while that of the second embodiment, FIG. 3B, depicts the deployed array element 26 as somewhat rectilinear. Both embodiments show that the deployed state of array element 26 is substantially perpendicular to the longitudinal axis of lead 23. The embodiment of stabilizing array 24 depicted in FIG. 3B, will be referred to as the (variation). As illustrated throughout, dissection line (C), taken across a distal segment of lead 23, will only depict distal electrode contact 25 when referencing dependent keeper 27(c) of FIGS. 9 and 10B.

One embodiment of stabilizing array 24, and said (variation), is keeper 27. Keeper 27 holds the distal tips of array element 26 in the stored (folded) position during percutaneous positioning of lead 23. A stored array element 26 is substantially parallel to the longitudinal axis and no wider than any epidural segment of lead 23. Keeper 27 is a static (non-movable) element and may be isolated (independent) or integral (dependent) with respect to distal electrode contact 25. Keeper 27 may be formed as a separate piece, or pieces, that are assembled together to form keeper 27 within lead 23. Alternatively, keeper 27 may be integrally formed as a single piece on insulating body 20, body 28 or a combination of the two.

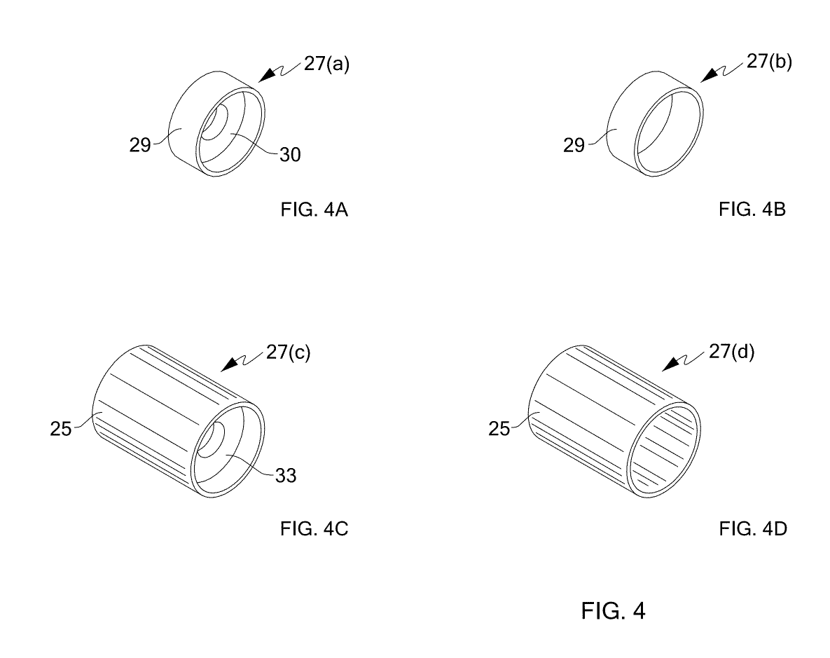

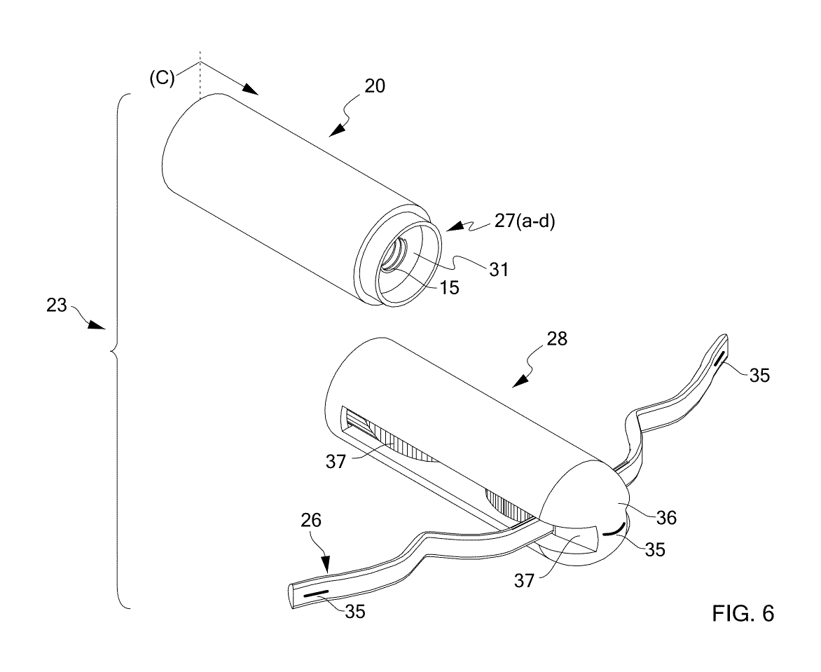

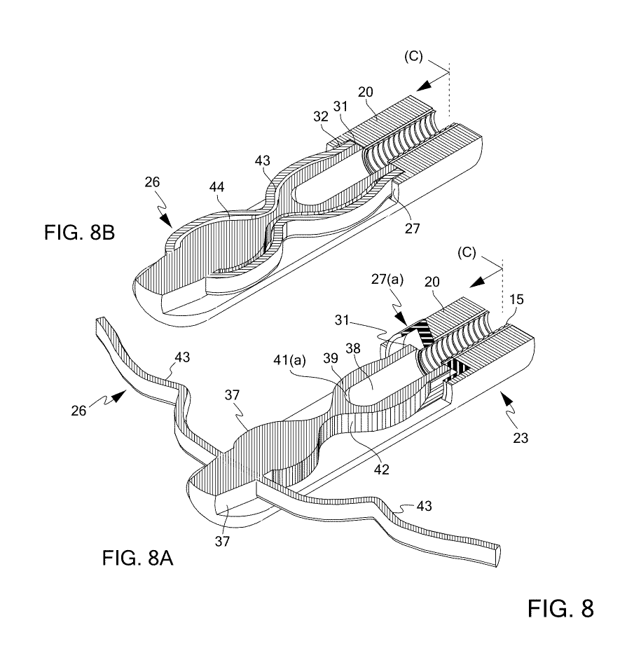

In accordance with the invention, FIGS. 4A and 4B are depictions of independent keepers 27(a/b) shown in perspective positions. In this embodiment, independent keepers 27(a/b) are electrically isolated as it places stabilizing array 24, and said (variation), distal to distal electrode contact 25. Independent keepers 27(a/b) must prevent shear stress failure of lead 23 when deployable array element 26 is stored. It is likely that independent keepers 27(a/b) will be manufactured from a polymer or metal such as, but not limited to, cross-linked polyurethane, MP35N super Alloy™, stainless, titanium or the like. By way of example but not limitation, independent keeper 27(a) is comprised of a ring 29 and collar 30. Collar 30 functions as backstop (31 of FIGS. 6 and 8), bonding surface and prevents electrical coupling with a potentially conductive stylet guide 15. It is likely that collar 30 will be a polymer such as, but not limited to, polyurethane as referenced by Kuzma (U.S. Pat. No. 7,891,085 B1). Backstop 31 prevents reward movement of array element 26 during percutaneous positioning of lead 23. The second independent keeper 27(b) eliminates collar 30 and relies on insulating body 20 for the functions of backstop 31, bonding surface and electrical decoupling.

In accordance with the invention, FIGS. 4C and 4D are depictions of dependent keepers 27(c/d) shown in perspective positions. In this embodiment, dependent keepers 27(c/d) are integral with distal electrode contact 25. As such, distal electrode contact 25 becomes part of stabilizing array 24 and said (variation). By way of example, but not limitation, dependent keeper 27(c) is comprised of distal electrode contact 25 and insulating disc 33. Insulating disc 33 functions as backstop 31, bonding surface and prevents electrical coupling with a potentially conductive stylet guide 15. It is likely that insulating disc 33 will be a polymer such as that used for collar 30. The second dependent keeper 27(d) eliminates insulating disc 33 and relies on insulating body 20 for the functions of backstop 31, bonding surface and electrical decoupling. For illustrative purposes only, distal electrode contact 25 is only shown in axial cutaway depictions (FIGS. 9 and 10B) referencing dependent keeper 27(c).

In yet another embodiment, keeper 27 may be integrally formed as a single piece if insulating body 20 and stabilizing array 24, including (variation) of, are formed concurrently. Alternatively, keeper 27 may be integrally formed on insulating body 20, or body 28, and then assembled (bonded) to complete stabilizing array 24 and said (variation). As an example, but not limitation, FIG. 8B, a perspective axial cutaway as taken from dissection line (C), shows a construct using a molded insulating body 20 serving as keeper 27, backstop 31 and bonding surface for body 28. The polymer selected for such a construct, generally at the level of keeper 27, or the entire lead 23, must prevent failure, of said lead, secondary to torsion and shear stress caused by the stored array element 26.

Keepers 27(a/c) require bonding (fusion and/or encapsulation) to insulating body 20, stylet guide 15, if different from insulating body 20, and body 28 if formed independently from insulating body 20. Stylet guide 15, if different from insulating body 20, will not require bonding to keepers 27(b/d) because said keepers lack collar 30 and insulating disc 33 features respectively.

Keeper 27 may be widely varied; for example, the surface in direct contact with the stored array element 26 may be smooth or include dents, slots, tabs or the like. These features allow keeper 27 to be configured for holding array element 26. An Independent keeper 27 may be widely varied; for example, the shape may be a ring or a more complex form with a snug fitting interface substantially similar around seating recess (32 of FIG. 9) such that array element 26 is substantially uniformly placed inside seating recess 32. Furthermore, a metallic keeper 27 may remain exposed or be encapsulated within and around seating recess 32 with a polymer substantially similar to insulating body 20, body 28, or a combination of the two.

As referenced by Cross (US patent 2006/0089692 A1), high tensile strength is required to enable non-stabilized cylindrical stimulator lead 12 to be reliably removed using a retrograde technique. Tensile strength is also relevant to lead 23 during the deployment of array element 26. With reference to Kuzma (U.S. Pat. No. 7,891,085 B1) an option to fill and possibly bond (fuse) empty wire conduit lumens 18, distal to the electrical wiring of stimulator electrode contacts 14, contributes to the tensile modulus of non-stabilized cylindrical stimulator lead 12. By way of inference but not limitation to lead 23, said filling and possible bonding will provide additional surface area for bonding keepers 27(a/c) and will seal lead 23 if insulating body 20 is used for backstop 31, as in keepers 27(b/d) or lead 23 with a integrally formed keeper 27.

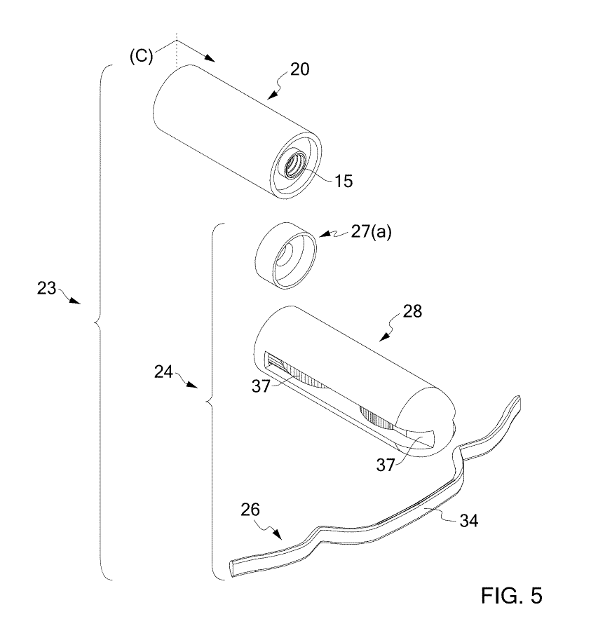

FIG. 5 shows a perspective exploded view of lead 23 as taken from dissection line (C). The fundamentals of Insulating body 20 and stylet guide 15 represent prior art. Wire conduit lumens 18 are not shown within insulation body 20. They may remain as open voids or can be filled and possibly bonded. Keeper 27(a) is exemplified in the exploded view and array element 26 is shown separated from body 28. In one embodiment of the invention, array element 26 can be individually formed from a medically implantable, non-resorbable, polymer such as, but not limited to, polyethylene, polyurethane or crossed-linked polyurethane. As an example, but not limitation, intrabody segment 34 of an independently formed array element 26 is embedded, by fusing or casting, within body 28. In another embodiment, array element 26, and body 28 can be concurrently formed if the polymer selected is substantially the same for array element 26 and body 28. With inference to keeper 27, the surface of array element 26 in direct contact with keeper 27 may be widely varied; for example, the surface may be smooth or include dents, slots, tabs or the like. These features allow array element 26 to be configured for secure storage juxtaposed to keeper 27.

As taken from dissection line (C), FIG. 6 shows a perspective exploded view of partially assembled lead 23 depicting keeper type 27(a-d) bonded to insulating body 20 and type specific bonded to stylet guide 15. When compared to FIG. 5, intrabody segment 34 of array element 26 is contained within body 28 where it is embedded as a separate component or was concurrently formed with body 28.

In still a further embodiment of the invention, radiopaque (x-ray) markers 35, which contrast radiolucent polymers, are likely to be integrated into, or formed around, a segment of array element 26. A radiopaque marker(s) 35 located substantially near or at distal tip 36, of body 28, would also be an option, especially if a radiopacifying element, or alloy, is not utilized for keeper 27. Radiopaque markers 35 provide fluoroscopic, x-ray, detection during percutaneous placement of lead 23 and deployment of array element 26. If removal of lead 23 is necessary, radiopaque markers 35 will assist the practitioner with respect to the location and extraction progress of lead 23 and its retroflexed array element (26 of FIG. 10) By way of example, polyurethane/Tungsten marker bands (Radiopaque Solutions Inc.) may be formed to array element 26 and/or a radiopacifying element, such as Tantalum, may be added to the monomer, prior to polymerization, of body 28 and/or array element 26.

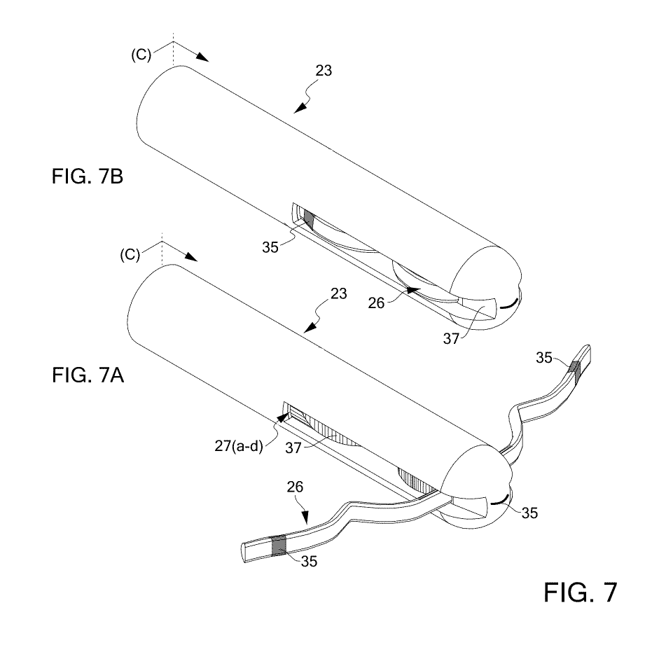

FIG. 7 shows the embodiment of stabilizing array 24 in a perspective view as taken from dissecting line (C). FIGS. 7A and 7B depict array element 26 in deployed and stored positions respectively. In yet another embodiment of stabilizing array 24, contours (37 of FIGS. 5-10) are formed on body 28 which generally follows the form of array element 26 in stored and retroflexed positions. For example, the spacing between array element 26 and contours 37 is substantially similar around the entire periphery of body 28, such that array element 26 is substantially uniformly stored, and retroflexed, abutting body 28. During percutaneous positioning and retrograde removal, contours 37 allow folded and retroflexed array element 26 to assume a dimension no wider than any epidural segment of lead 23.

The embodiments of stabilizing array 24, as discussed and illustrated in FIGS. 5-7, are substantially the same for the (variation) in stabilizing array 24. Axial cutaway depictions (FIGS. 8 and 9) highlight the internal differences between stabilizing array 24 and said (variation).

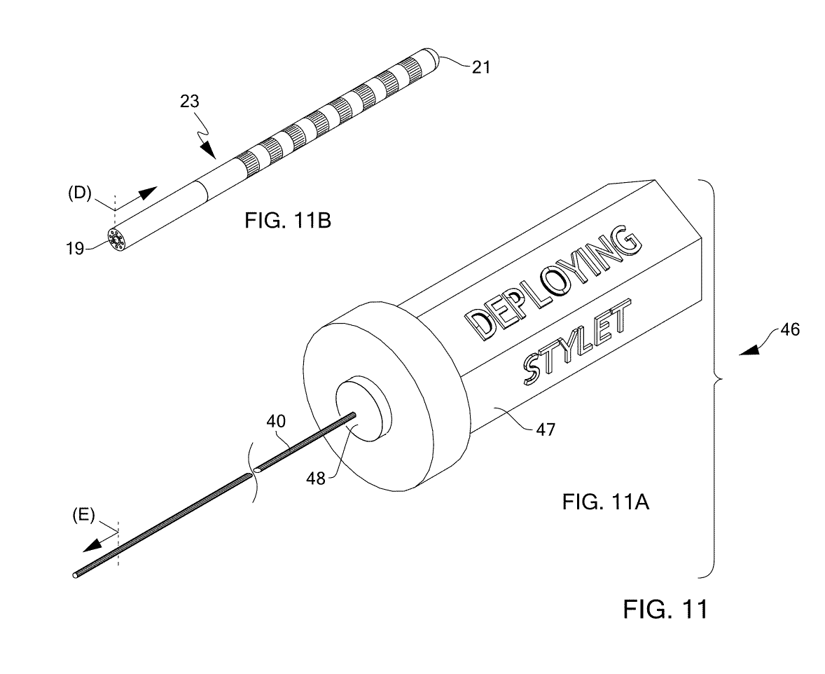

Deploying lumen (38 of FIGS. 8-10 and 12) is yet another embodiment of stabilizing array 24 and said (variation). Deploying lumen 38, continuous with coaxial lumen 19, accommodates the deploying segment, dissecting line (E), of deploying stylet 40 of FIGS. 11-14. Deploying lumen 38 generally originates at the level of backstop 31, may be sized to be substantially equal to stylet guide 15, has a length dependent on stabilizing array 24, and said (variation), and may have a substantially flat or rounded luminal contact surface 41 such that the distal tip of deploying stylet 40 is substantially uniformly matched to luminal contact surface 41. Sidewalls 39, surrounding deploying lumen 38, may have an embedded wire wound feature (not shown) generally originating and terminating at backstop 31 and luminal contact surface 41 respectively.

FIG. 8 details the embodiment of stabilizing array 24 in a perspective axial cutaway representation, as taken from dissection line (C). Distal electrode contact 25 is not shown as it is not integral with the depicted independent keeper 27(a). Stylet guide 15 is specific to manufacturing and, for illustrative purposes, is shown as a coiled wire feature. To provide detail, one side of keeper 27(a) is shown elevated out of the axial cutaway depiction of FIG. 8A. In the illustrated embodiment, deploying lumen 38 terminates at luminal contact surface 41(a) which is contiguous with contoured deploying surface 42. Juxtaposed to contoured deploying surface 42 is deploying contact surface 43 of the curvilinear shaped array element 26. By way of example but not limitation, the embodiment of stabilizing array 24 depicts relief contour 44 distal to, and substantially mirroring, contoured deploying surface 42. As seen in FIG. 8B, relief contour 44 accommodates the deflection of array element 26 forward progresses during packing (storage) and deployment.

FIG. 9 details the (variation) in stabilizing array 24 in a perspective axial cutaway representation, as taken from dissection line (C). Keeper 27(c), integral with distal electrode contact 25, is shown partially elevated out of the axial cutaway illustration. The (variation) in stabilizing array 24, which utilizes a substantially rectilinear deployed array element 26, has deploying lumen 38 terminating at luminal contact surface 41(b); notably isolated from the deployable surfaces of array element 26.

Another purpose of this invention is to provide a safe means of lead removal in the event of lead failure, infection or medical and/or patient necessity. An intact, non-stabilized cylindrical lead 12 can be removed by a retrograde technique (prior art). In yet another embodiment of the invention, deployed array element 26 has the ability to fold back on itself (retroflex). Retroflexing of array element 26 upholds the practice of retrograde removal for lead 23.

Illustrating retroflexed array elements 26: FIGS. 10A and 10B, perspective views as taken from dissection line (C), show stabilizing array 24, and said (variation), in whole and axial cutaway depictions respectively. FIG. 10B depicts keeper 27(c) which is integral with the distal electrode contact 25. As previously noted, contours 37 allow array element 26 to achieve a stored and retroflexed dimension no wider than any epidural segment of lead 23. Additionally, the polymer selected for array element 26 may require an intrinsic perforation, thinning or retroflexing relief cut 45 to achieve an optimal retroflexed dimension for retrograde extraction of lead 23.

The method (prior art) of percutaneously implanting non-stabilized cylindrical lead 12 is well documented. Except for the unique deployment of array element 26, the basic implantation steps of non-stabilized lead 12 apply to lead 23. Those basic steps involved with implanting lead 23 (within the epidural space 103) as well as the unique step of deploying array element 26 will now be discussed in further detail.

The practitioner identifies the vertebral level to be entered for percutaneous placement of lead(s) 23. Using sterile technique a percutaneous introducer needle i.e. Tuohy or Hustead (108 of FIG. 2A), is inserted using a paramedian or midline approach. With tactile feedback and possible fluoroscopic or ultrasonic assistance, the introducer needle 108 is advanced through the supraspinous ligament 100 and into the intraspinous ligament 101 for a midline approach to the epidural space 103 or to the lamina 116 for a paramedian approach to the epidural space 103. The introducer needle stylet 110 is removed, which prevents coring of soft tissue, and the introducer needle tip 111 is advanced into the epidural space 103 through the ligamentum flavum 102 using the traditional loss of resistance technique with air or sterile saline. The introducer needle 108 can be rotated so that needle tip 111, with its beveled cutting surface, aims in the direction of catheter advancement, i.e. cephalad, prior to, or after, the advancement of the introducer needle 108 into the epidural space 103. Using fluoroscopic guidance, lead 23 is inserted through the lumen of the introducer needle 108 and advanced to the targeted stimulation site within the epidural space 103. Guiding stylet 16, introduced into proximal originating coaxial lumen 19, may be required to stiffen and steer lead 23 to obtain final positioning of electrode array 13. Assessment of electronic integrity, which confirms continuity of electrode array 13, electrical conductor wires 17 and wired contacts 22, is commonly preformed when lead 23 is at, or near, its final location within the epidural space 103. A practitioner's preference determines if intra-operative stimulation testing, using a non-implantable pulse generator (not shown), is preformed in a responsive (awake) patient. Such testing optimizes initial stimulator performance and offers the chance of fine tuning electrode array 13 positioning over the targeted dorsal column 117 of the spinal cord 107. Using fluoroscopic guidance, guiding stylet 16 is carefully removed from coaxial lumen 19 to prevent movement of the epidurally implanted segment of lead 23.

For clarification, guiding stylet 16, comprised of a wire sized to fit within the proximally originating coaxial lumen 19, is shorter than deploying stylet 40 and will not enter deploying lumen 38.

By way of example but not limitation, deploying stylet 40 may be of similar material and sized to be substantially equal to the width (gauge) of guiding stylet 16. Furthermore, a step-down radius in the deployment section of deploying stylet 40 may be necessary to prevent binding of said stylet with sidewalls (39 of FIGS. 8, 9 and 12) of deploying lumen 38. The distal tip of deploying stylet 40 may have a substantially flat or rounded distal tip which is substantially uniformly matched to luminal contact surface 41. The lengths of deploying stylet 40 are, to some extent, dependent on stabilizing array 24 and said (variation).

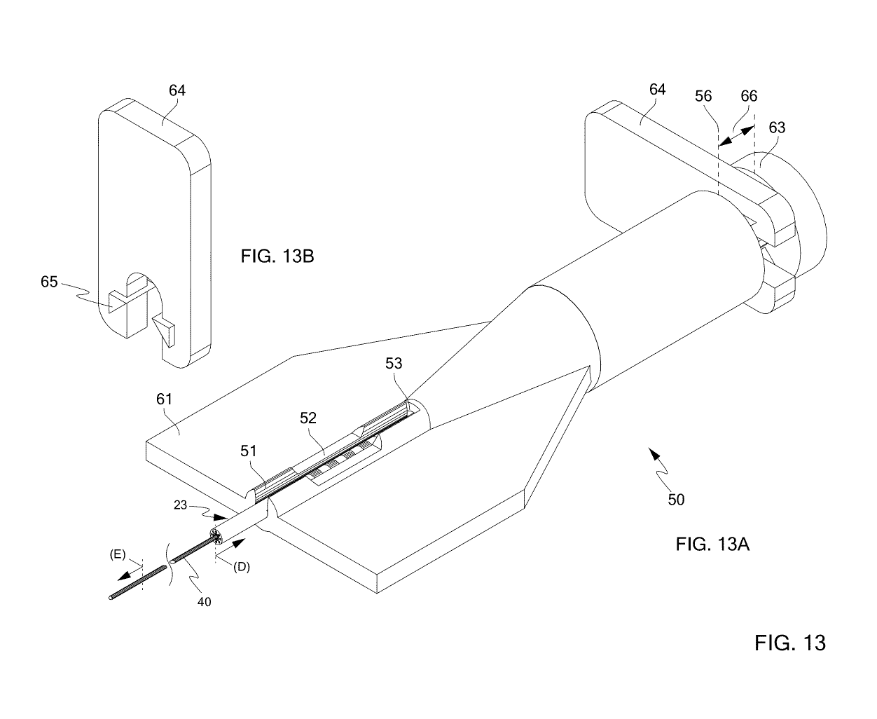

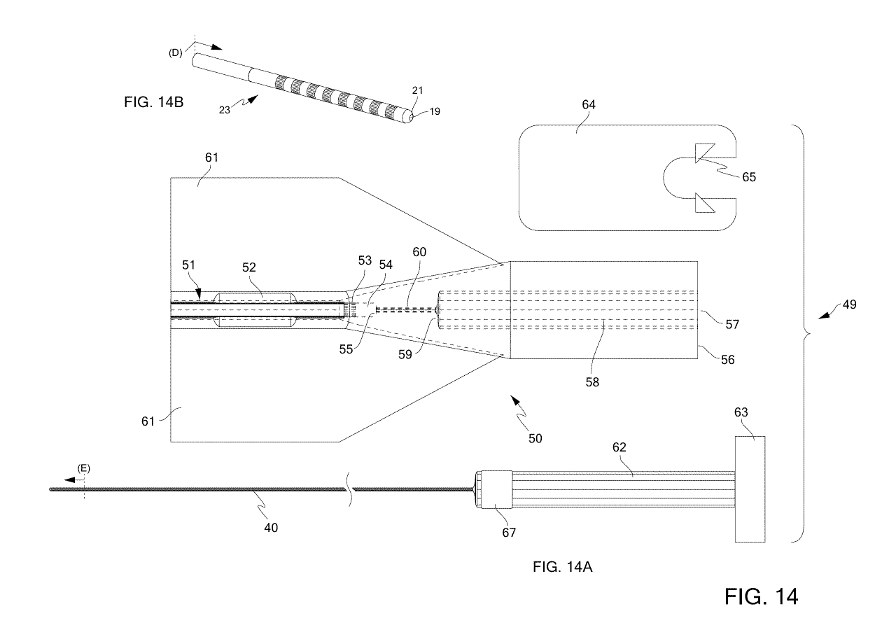

Deployment of array element 26 occurs after fluoroscopic assisted final positioning of lead 23 and possible electrode array 13 stimulation testing in a responsive patient. Deployment of array element 26 is done with either stylet (46 of FIG. 11A) or deploying handpiece (49 of FIGS. 13 and 14A). The embodiments and deployment methods of each will now be illustrated and discussed in detail.

FIGS. 11A and 11B depict the embodiment of stylet 46 and a proximal segment of lead 23, as taken across dissection line (D), respectively. Control stop 48 on deploying stylet body 47, which may be molded plastic, contacts proximal tip 21 of lead 23 thereby preventing the deploying segment of deploying stylet 40 from displacing stabilizing array 24, and said (variation), any further than necessary to deploy array element 26. Stylet 46 must be clearly identified to prevent its use during percutaneous guiding and final positioning of lead 23.

There exists a potential for inductive movement of electrode array 13, possibly away from the targeted stimulation site, during the deployment of array element 26 when using stylet 46. Inductive movement is attenuated by countertraction between the extra-epidural segment of lead 23 and deploying stylet body 47 of stylet 46.

With final epidural positioning of lead 23 complete and fluoroscopic guidance present, deployment of array element 26 using stylet 46 is accomplished in the following sequence: deploying stylet 40 is advanced through proximally originating coaxial lumen 19; prior to deployment, countertraction is established and maintained as deploying stylet 40 is advanced into deploying lumen 38; deployment of array element 26 initiates as the distal tip of deploying stylet 40 seats to luminal contact surface (41(a/b) of FIGS. 8A and 9); continued advancement of deploying stylet 40 elastically elongates body (28 of FIG. 6) and displaces the retained tips of array element 26 from keeper 27; deployment of array element 26 concludes when control stop 48 contacts proximal tip 21 of lead 23 whereby array element 26 assumes its permanent (intrinsic) deployed shape.

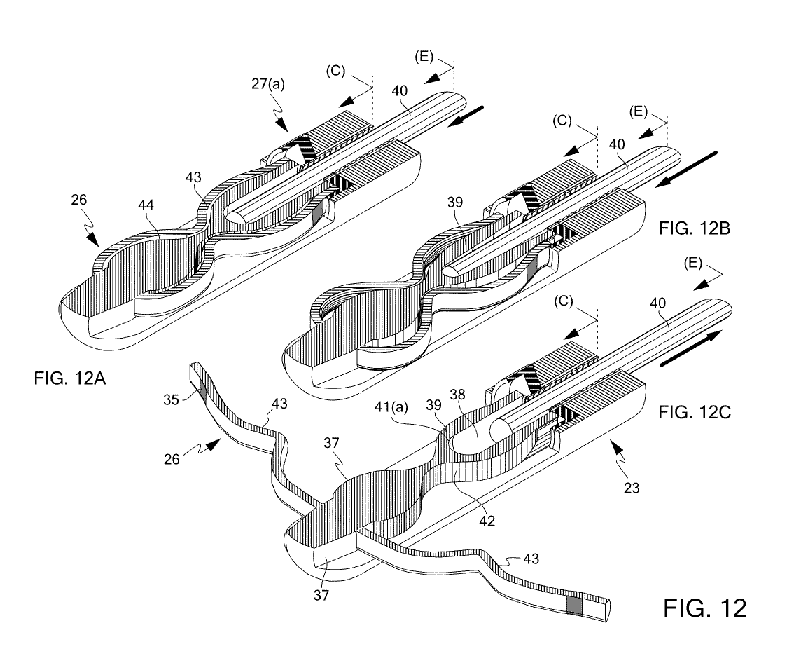

To reduce tensile stress on lead 23, deploying stylet 40 is carefully pulled away from deploying lumen (38 of FIG. 12C) and positioned within the epidurally implanted segment of lead 23. As noted below, removal of deploying stylet 40 occurs after fluoroscopic verification of array element 26 deployment, rotational alignment and extraction of the percutaneous introducer needle 108.

The present invention relates, in yet another embodiment, to deploying handpiece 49 comprised primarily of: a handpiece 50; a deploying stylet 40; a plunger 62; and a safety tab 64. The embodiment of deploying handpiece 49, as illustrated in FIGS. 13 and 14A, will now be described in more detail.

A proximal segment of lead 23, which may included all wired contacts 22, is secured by deploying handpiece 49 and eliminates the manual countertraction necessary on the extra-epidural segment of lead 23 during deployment of array element 26. Deploying handpiece 49 incorporates deploying stylet 40 on to plunger 62 rather than deploying stylet body (47 of FIG. 11A). Deploying handpiece 49 is intended for the deployment of array element 26. While not a replacement for guiding stylet 16, lead 23 can be guided within the epidural space 103 using deploying handpiece 49 with safety tab 64 secured to plunger 62.

FIG. 13A is a perspective view of deploying handpiece 49. Safety tab 64 is shown clipped to plunger (62 of FIG. 14). A proximal segment of lead 23, as taken from dissection line (D), is depicted in retention feature 51. Projecting beyond dissection line (D), a segment of deploying stylet 40 is shown within the proximally originating coaxial lumen 19.

FIG. 13B details plastic safety tab 64 with integral locking clips 65. Locking clips 65 maintain non-deployable distance 66 between plunger finger rest 63 and cylindrical end 56 of handpiece 50. Non-deployable distance 66 prevents deploying stylet 40 from entering deploying lumen 38. Furthermore, locking clips 65 securely fasten safety tab 64 to plunger 62. Removal of safety tab 64 can only happen with a pulling and/or twisting action.

FIG. 14A is a top view of deploying handpiece 49. As a reference, FIG. 14B depicts a proximal segment of lead 23 as taken across dissection line (D). FIG. 14A shows the embodiment of handpiece 50 in a dashed hidden line format to illustrate its internal structure. The embodiment of handpiece 50, which may be molded plastic, is somewhat wing like and comprises: a retention feature 51; a recess 52; a tapered relief 53; a cavity 54; a seating surface 55; a cylindrical end 56; a cylindrical opening 57 for plunger 62; a cylinder 58; a cylinder floor 59; a stylet passage way 60; and opposing tabs 61. Of course, this is not a limitation. For example; handpiece 50 may have a more complex shape and/or include such additions as finger seats to aid in the downward displacement of plunger 62 and/or a stylet passageway 60 with a wire wound element substantially similar to stylet guide 15 of non-stabilized lead 12 or lead 23. Retention feature 51 is formed to match the outer shape of lead 23 with an interface configured for a snug fit. Retention feature 51 can be widely varied; it may, for example, include slots, tabs, snaps or the like. Stylet passageway 60 links cylinder floor 59 to seating surface 55. As an example, but not limitation, the diameter of stylet passageway 60 is generally comparable to coaxial lumen 19 of lead 23. This prevents bending and possible failure of deploying stylet 40 during deployment of array element 26. Plunger 62, integral with deploying stylet 40, may contain flat o-ring 67 which provides a smooth gliding surface between plunger 62 and cylinder 58.

By way of example, and with deploying handpiece 49 fully assembled, safety tab 64 attached to plunger 62, and fluoroscopic guidance present, the loading of a proximal segment of lead 23 into deploying handpiece 49 takes place in the following sequence: deploying stylet 40 is advanced through proximally originating coaxial lumen 19; tapered relief 53 allows proximal tip 21 to be angled and inserted into cavity 54 where it is mated to seating surface 55; retention feature 51 is opened by a flexing action of tabs 61; recess 52, shown generally gaping retention feature 51, allows lead 23 to be finger pressed (seated) into retention feature 51; after confirming that proximal tip 21 is mated to seating surface 55, tabs 61 are released trapping a proximal segment of lead 23. Failure to seat proximal tip 21 to seating surface 55 may result in a null deployment of array element 26.

With a proximal segment of lead 23 loaded into deploying handpiece 49, and final epidural positioning of lead 23 complete, deployment of array element 26 for stabilizing array 24 and said (variation) is accomplished by removing safety tab 64 and pressing plunger finger rest 63 until plunger 62 bottoms out on cylinder floor 59. The action of pressing said plunger seats stylet 40 to luminal contact surface (41(a/b) of FIGS. 8A and 9) elastically elongates body (28 of FIG. 6) and displaces the retained tips of array element 26 from keeper 27. Bottoming out also prevents the deploying segment of deploying stylet 40 from displacing stabilizing array 24 and said (variation) any further than necessary to deploy array element 26 to its permanent (intrinsic) shape.

To reduce tensile stress on lead 23, deploying stylet 40 is carefully pulled away from deploying lumen (38 of FIG. 12C) and positioned within the epidurally implanted segment of lead 23.

Verification of array element 26 deployment and its rotational alignment within the epidural space 103 is confirmed by the fluoroscopic positional relationship of radiopaque markers 35 on array element 26 to the cylindrical portion of lead 23 with its radio-dense electrode array 13, optional distal tip 36 radiopaque marker(s) 35 and contrasting radiolucent polymer insulating body 20. A correctly positioned stabilizing array 24, and said (variation), will image with the bilateral radiopaque markers 35 of array element 26 extended and substantially perpendicular to the longitudinal axis of lead 23 as viewed from an anterior/posterior fluoroscopic image. As previously noted, the epidural space 103 is a potential space with major borders consisting of dural sac 112, ligamentum flavum 102, and the vertebral pedicals 115 and laminas 116. As the array element is deployed it will follow the path of least resistance and will rotate away from the borders of the epidural space 103. If necessary, the extra-epidural segment of lead 23, generally at the level of the introducer needle hub 109, can be carefully twisted until the acquired image of two radiopaque markers 35 of array element 26 are obtained.

After confirmed deployment of array element 26, removal of the retained proximal segment of lead 23 from deploying handpiece 49 is accomplished by a flexing action of tabs 61 allowing lead 23 to be angled and carefully pulled free of retention feature 51 and cavity 54.

The prior art of: removing the introducer needle 108 and guiding stylet 16 (with inference to deploying stylet 40); anchoring the extra-epidural segment of non-stabilized lead 12 as it emerges from the epidural space 103; adding possible lead extensions; soft tissue tunneling of said lead and/or lead extensions to the implantable pulse generator 11 implant site; establishing electronic connections, testing and initial programming of said lead and pulse generator; implantation of said pulse generator; and surgical closures of anchoring and implant sites—are relevant and generally apply to lead 23.

To elucidate the prior art of introducer needle 108 and guiding stylet 16 removal as it applies to lead 23 and deploying stylet 40 the following sequence is performed: the extra-epidural anchoring and tunneling site is surgically prepared; fluoroscopy is used to visualize deploying stylet 40 and the positional stability of electrode array 13 during deploying stylet 40 removal and extraction of the introducer needle 108; deploying stylet 40 is partially withdrawn—locating its distal tip generally at the beveled tip 111 of the introducer needle 108; to attenuate movement of the epidurally implanted segment of lead 23, minimal traction is used to remove the introducer needle 108 from surrounding tissue and the remainder of deploying stylet 40 is carefully removed from coaxial lumen 19.

While the invention has been described in terms of several preferred embodiments, numerous alterations, permutations and equivalents could be made thereto by those skilled in the art without departing from the scope of the invention. It is therefore intended that the following claims be interpreted as including all such alterations, permutations and equivalents as fall within the true spirit and scope of the present invention.

I claim:

1. A stabilizing array comprising:
a body;
an array element joined to said body;
said array element capable of folding to abut said body when said stabilizing array is in a stored position;
said array element capable of unfolding to extend laterally from said body when said stabilizing array is in a deployed position.

2. The stabilizing array of claim 1, wherein said body further comprises:
a proximal end and a distal end;
a deploying lumen that opens at said proximal end for receiving a deploying stylet;
a seating recess for holding said array element in a stored position; and
recesses on opposite sides of said body;
said array element substantially matching said recesses such that said array element is capable of folding into said recesses and held adjacent to said body when said stabilizing array is in said stored position.

3. The stabilizing array of claim 2, wherein said recesses on opposite sides of said body are contoured to match the shape of said array element.

4. The stabilizing array of claim 1, wherein said array element consists of two arms, said array element being capable of retroflexing such that said arms fold back by extending away from said body.

5. The stabilizing array of claim 1, wherein said array element is curvilinear.

6. The stabilizing array of claim 1, wherein said array element is rectilinear.

7. A device for medical implant, said device comprising:
a deploying stylet;
a stabilizing array capable of being deployed by said deploying stylet, said stabilizing array comprising:
a body with an array element;
a keeper for holding said array element in a stored position;
wherein said array element is folded to abut said body when said stabilizing array is in said stored position; and,
wherein said array element is extended laterally from said body when said stabilizing array is deployed by said deploying stylet to release said array element from said keeper.

8. The device of claim 7, wherein said body of said stabilizing array has a contoured recess for receiving said array element when said array element is folded in said stored position.

9. The device of claim 8, wherein said array element is folded into said contoured recess of said body such that said stabilizing array is no wider than any epidural segment of said stimulator lead when said array element is in said stored position.

10. The device of claim 7, wherein said array element consists of two arms joined by an intrabody segment, said intrabody segment embedded in said stabilizing array body, said arms having distal tips retained by said keeper such that said arms are folded to abut said body when said stabilizing array is in said stored position.

11. The device of claim 7, wherein said stabilizing array has a deploying lumen for receiving said deploying stylet.

12. The device of claim 11, wherein said array element is deployed by advancing said deploying stylet into said deploying lumen to elongate said body of said stabilizing array which causes said array element to be released from said keeper.

13. The device of claim 7, said keeper further comprising: a ring body.

14. The device of claim 7, said keeper further comprising: a ring body, a collar within said ring body, wherein said collar functions to prevent rearward movement of said array element and electrical coupling with said deploying stylet.

15. The device of claim 7, said keeper further comprising: a distal electrode contact.

16. The device of claim 7, said keeper further comprising: a distal electrode contact, an insulating disc within said distal electrode contact, wherein said insulating disc functions to prevent electrical coupling with said deploying stylet.

17. The device of claim 7, wherein said keeper includes a dent, slot, or tab for holding said array element.

18. The device of claim 7, wherein said keeper includes a seating recess for holding said array element in said stored position.

19. The device of claim 6, wherein said array element includes at least one x-ray marker.

20. A device for medical implant, said device comprising:
a deploying stylet;
a stabilizing array capable of being deployed by said deploying stylet, said stabilizing array comprising:
a body, said body having a deploying lumen for receiving said deploying stylet;
an array element, said array element having distal tips retained in a seating recess of said body such that said array element folds to abut said body when said stabilizing array is in a stored position;
wherein said array element is deployed by pushing said deploying stylet into said deploying lumen to elastically elongate said body of said stabilizing array such that said array element is released from said seating recess.

21. A handpiece for deploying a stabilizing array, said handpiece comprising:
a body, said body having a recess with a retention feature for holding a stimulator lead;
a plunger for deploying a stabilizing array;
a safety tab having locking clips;
wherein said safety tab is interposed between said plunger and said body to prevent depression of said plunger to deploy said stabilizing array.

22. A method for deploying a stabilizing array, said stabilizing array having an array element joined to a body, said array element capable of folding to abut said body when said stabilizing array is in a stored position and unfolding to extend laterally from said body when said stabilizing array is in a deployed position, said method comprising:
inserting a deploying stylet into a lumen passage of said stabilizing array until said body of said stabilizing array is elastically elongated to release said array element from said stored position.
